(19) United States
(12) Patent Application Publication
(10) Pub. No.: US 2020/0054495 A1
(43) Pub. Date: Feb. 20, 2020

(54) UNDERGARMENT HYGIENE PRODUCT

(71) Applicant: Lizle Pelt, Colorado Springs, CO (US)

(72) Inventor: Lizle Pelt, Colorado Springs, CO (US)

(21) Appl. No.: 16/371,786

(22) Filed: Apr. 1, 2019

Related U.S. Application Data
(60) Provisional application No. 62/718,790, filed on Aug. 14, 2018.

(51) Int. Cl. A61F 13/496; A61F 13/56; A61F 13/15; A61L 15/26; A41B 9/00

(52) U.S. Cl. CPC A61F 13/496; A61F 13/5616; A61F 13/15268; A61F 2013/4708; A41B 9/002; A61F 2013/15121; A61L 15/26

(57) ABSTRACT

An absorbent article for use as an undergarment hygiene product comprises: a first portion having an elliptical shape and a first width; a connecting portion extending from the first portion and having a second width; a second portion extending from the connecting portion and having a third width, the third width greater than the second width; and, a fastening system arranged on an outer surface of the absorbent article for fixing the absorbent article to an inner surface of an article of clothing. Another absorbent article for use as an undergarment hygiene product comprises: a longitudinal axis, a first portion having an elliptical shape and a first width symmetrical about the longitudinal axis; a connecting portion extending from the first portion and having a second width; a second portion extending from the connecting portion and having a third width, the third width greater than the second width; a fastening system arranged on an outer surface of the absorbent article for fixing the absorbent article to an inner surface of an article of clothing: and, wherein the second portion includes a middle section including the longitudinal axis and having an edge perpendicular to the longitudinal axis, the middle section between first and second wing sections, the first and second wing sections symmetrical about the longitudinal axis.

### FIELD OF THE DISCLOSURE

The present disclosure relates generally to absorbent garments for adult incontinence, and more specifically, to undergarment hygiene products that are disposable and/or renewable and methods for using such undergarment hygiene products.

### BACKGROUND

Undergarment hygiene products such as adult incontinence products and the like typically are bulky, uncomfortable, and are not concealable under underwear or clothing styles that are pleasing to the wearer. In addition, absorbent garments are generally disposable contributing to waste and harmful for the environment.

### SUMMARY

Accordingly, there is a need for an undergarment hygiene device, method and/or system that has the capability to be secured to a wearer's clothing in concealing manner, as well as to be renewed or disposed according to the preference of the wearer. The present disclosure discusses a method and device that satisfies such needs.

An article in accordance with this disclosure includes an absorbent article for use as an undergarment hygiene product, the absorbent article comprising: a first portion having an elliptical shape and a first width; a connecting portion extending from the first portion and having a second width; a second portion extending from the connecting portion and having a third width, the third width greater than the second width; and, a fastening system arranged on an outer surface of the absorbent article for fixing the absorbent article to an inner surface of an article of clothing.

The article may also or alternatively include wherein the fastening system includes adhesives.

The article may also or alternatively include wherein the fastening system includes buttons.

The article may also or alternatively include wherein the absorbent article is disposable.

The article may also or alternatively include wherein the absorbent article is renewable.

The article may also or alternatively include wherein the absorbent article is made of cotton.

The article may also or alternatively include wherein the connecting portion includes a first length l₁, the first length ranging from 1.5″ to 2.5″.

The article may also or alternatively include wherein the absorbent article includes a second length l_{T}, the second length ranging from 7″ to 10″.

The article may also or alternatively include wherein the absorbent article includes a width w_{T}, the width ranging from 3″ to 8″.

The article may also or alternatively include wherein the first portion is circular and includes a diameter, the diameter ranging from 3″ to 5″.

The article may also or alternatively include wherein the article of clothing is underpants.

Another article in accordance with this disclosure includes an absorbent article for use as an undergarment hygiene product, the absorbent article comprising: a longitudinal axis, a first portion having an elliptical shape and a first width symmetrical about the longitudinal axis; a connecting portion extending from the first portion and having a second width; a second portion extending from the connecting portion and having a third width, the third width greater than the second width; a fastening system arranged on an outer surface of the absorbent article for fixing the absorbent article to an inner surface of an article of clothing: and, wherein the second portion includes a middle section including the longitudinal axis and having an edge perpendicular to the longitudinal axis, the middle section between first and second wing sections, the first and second wing sections symmetrical about the longitudinal axis.

The article may also or alternatively include wherein the edge is flat or curved.

The article may also or alternatively include wherein the wing sections include the fastening system.

The article may also or alternatively include wherein the wing sections are tapered.

The article may also or alternatively include wherein the second portion includes a modified classic brief shape.

The article may also or alternatively include wherein the second portion includes a modified high-cut brief shape.

The article may also or alternatively include wherein the second portion includes a modified hipster brief shape.

The article may also or alternatively include wherein the second portion includes a modified bikini brief shape.

The article may also or alternatively include wherein the second portion includes a modified boyshorts brief shape.

The article may also or alternatively include wherein the second portion includes a modified tanga brief shape.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

The term “a” or “an” entity refers to one or more of that entity. As such, the terms “a” (or “an”), “one or more” and “at least one” may be used interchangeably herein. It is also to be noted that the terms “comprising”, “including”, and “having” may be used interchangeably.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure may be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a top view of an embodiment of an absorbent article of the present disclosure;

FIG. 2 is a bottom view the embodiment as shown in FIG. 2;

FIG. 3 is a top view of an alternative embodiment of an absorbent article of the present disclosure;

FIG. 4 is a top view of an alternative embodiment of an absorbent article of the present disclosure;

FIG. 5 is a top view of an alternative embodiment of an absorbent article of the present disclosure;

FIG. 6 is a top view of an alternative embodiment of an absorbent article of the present disclosure; and,

FIG. 7 is a top view of an alternative embodiment of an absorbent article of the present disclosure.

FIG. 8A is a perspective view of an embodiment of an absorbent article of the present disclosure having multiple material layers.

FIG. 8B is a cross-sectional view of the absorbent article of FIG. 8A.

FIG. 8C is a cross-section view of another embodiment, which is disposable, of an absorbent article of the present disclosure.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

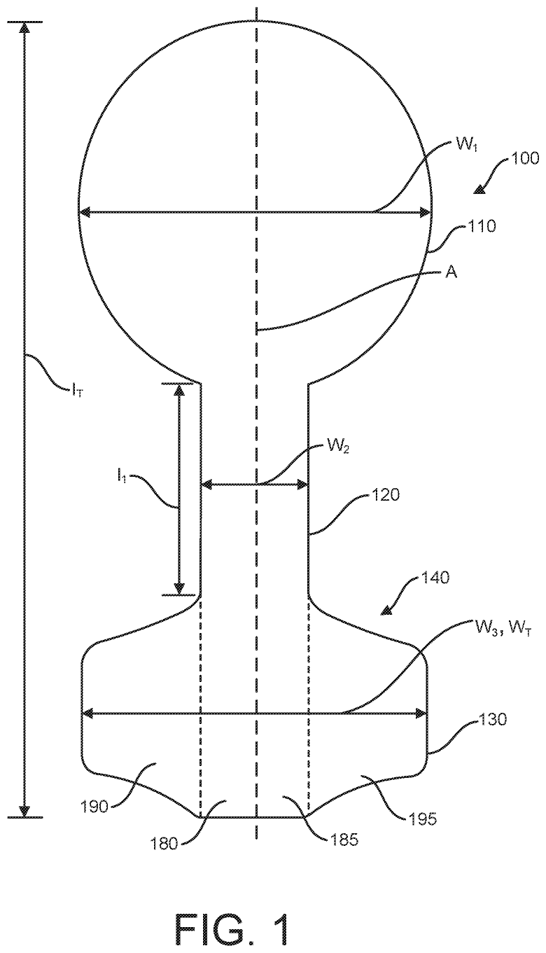

Embodiments according to this disclosure provide an absorbent article that can be worn discretely under the users own preferred fashionable attire. Specifically, embodiments according to this disclosure provide an absorbent article that can be worn discretely as well as being renewable or disposable. FIG. 1 depicts an absorbent article 100 for use as an undergarment hygiene product. Absorbent article 100 is generally to be worn adjacent to the user's skin and may be affixed to an article of clothing, as in some embodiments according to the present disclosure, such as underpants. Absorbent article 100 of the present disclosure may be made of any suitable absorbent material that absorbs and retains moisture. In some embodiments according to this disclosure, the absorbent article 100 is made of cotton. In other embodiments, the absorbent article 100 is made from suitable plastics such as water-absorbing polymers.

In exemplary embodiments, absorbent article 100 includes a first portion 110 having an elliptical shape and a first width w₁. Absorbent article 100 includes a connecting portion 120, having a second width w₂, extending from the first portion 110. Second portion 130 extends from the connecting portion 120 and has a third width w₃, wherein the third width w₃ is greater than the second width w₂. Additionally or alternatively, the first width w₁ may be greater than the second width w₂. The third width w₃ and/or the first width w₁ being greater than the second width w₂ increases comfort as the absorbent article extends from the anterior to the posterior of a person's undergarments. In addition, the increased first width w₁ and/or the increased third width w₃ reduces the likelihood moisture from incontinence will seep and/or circumvent the absorbent article 100. Furthermore, the increased first width w₁ and/or the increased third width w₃ may reduce the likelihood the absorbent article shifts while being worn and affixed to an article of clothing.

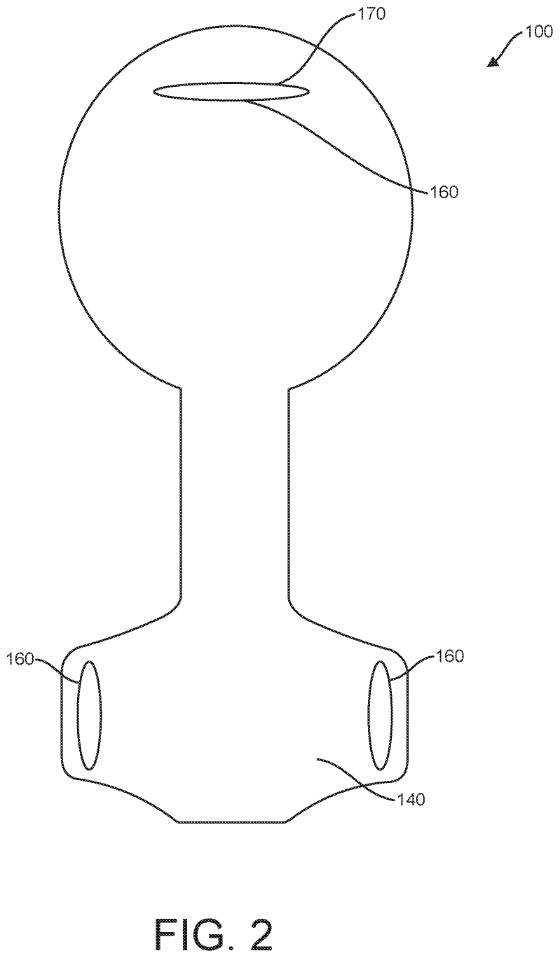

As shown in FIG. 2, absorbent article 100 includes a fastening system 160 arranged on an outer surface 140 of the absorbent article for fixing the absorbent article to an inner surface (not shown) of an article of clothing. Outer surface 140 is opposite inner surface 150 of absorbent article 100. In some embodiments according to this disclosure, fastening system 160 includes adhesives 170. FIG. 2 shows a non-limiting example of the potential placement of adhesives 170. The number and size of adhesives may be arranged to provide security of the absorbent article 100 so that the article is inhibited from movement during use. Adhesives 170 are placed strategically to maintain comfort of the article, and so as not to interfere with absorbency in critical locations (i.e. along the longitudinal axis A). The number and placement of adhesives may vary as needed based upon the overall size and shape complexity of the article. In an alternative embodiment according to this disclosure, absorbent article 100 includes a fastening system 160 that includes buttons, snaps, or other fastening means.

In some embodiments according to this disclosure, the absorbent article 100 is disposable, intended for one-time use, and may be made from suitable plastics such as water-absorbing polymers. A fastening system 160 using adhesives 170 strategically placed in the article is suitable for disposable absorbent articles 100 according to the present disclosure. In other embodiments according to this disclosure, the absorbent article 100 is renewable; in other words, absorbent article 100 may be cleaned, washed, or otherwise made hygienic in order to reuse for more than one use. Absorbent article 100 includes a fastening system 160 having buttons, snaps, or other fastening means are suitable for renewable absorbent articles 100 according to the present disclosure.

As shown in FIG. 1 and according to some embodiments according to this disclosure, absorbent article 100 for use as an undergarment hygiene product includes a longitudinal axis A and a first portion 110 having an elliptical shape and a first width w₁ symmetrical about the longitudinal axis A. In some embodiments according to this disclosure, the absorbent article 100 may include wherein the first portion 110 is circular and includes a diameter, the diameter ranging from about 3.5″ to about 5.5″. In other embodiments, the first portion 110 may have a non-elliptical shape while still having a greater first width w₁ than the second width w₂. First portion 110 includes a width w₁, the width w₁ ranging from about 3″ to about 5″. The width w₁ may be about 3.5″, about 4.0″, about 4.5″, about 5.0″, about 5.5″, in some embodiments, the width w₁ is from about 3.5″ to about 5.5″, or from about 4.0″ to about 5.0″.

As shown in FIG. 1, absorbent article 100 has connecting portion 120, which includes a first length l₁. First length l₁ ranges from about 1.5″ to about 2.5″. The first length l₁ may be about 1.5″, about 1.6″, about 1.7″, about 1.8″, about 1.9″, about 2.0″, about 2.1″, about 2.2″, about 2.3″, about 2.4″, about 2.5″, in some embodiments, the first length l₁ is from about 1.5″ to about 2.5″, from about 1.7″ to about 2.5″, or from about 1.9″ to about 2.1″. Connecting portion 120 includes a second width w₂ extending from the first portion 110. Connecting portion 120 includes a width w₂, the width w₂ ranging from about 0.5″ to about 1.5″. The width w₂ may be about 0.5″, about 0.75″, about 1.0″, about 1.25″, about 1.5″, in some embodiments, the width w₁ is from about 0.5″ to about 1.5″, or from about 0.75″ to about 1.25″.

Absorbent article 100 includes second (or total) length l_{T}. Second length l_{T} ranges from about 6.0″ to about 8.5″. The second length l_{T} may be about 6.0″, about 6.5″, about 7.0″, about 7.5″, about 8.0″, about 8.5″, in some embodiments, the second length l_{T} is from about 6.0″ to about 8.5″, from about 6.5″ to about 8.0″, or from about 7.0″ to about 8.0″. Absorbent article 100 includes a total width w_{T}, the width ranging from about 3″ to about 8″. The width w_{T} may be about 3.0″, about 3.5″, about 4.0″, about 4.5″, about 5.0″, about 5.5″, about 6.0″, about 6.5″, about 7.0″, about 7.5″, about 8.0″, in some embodiments, the width w_{T} is from about 3.0″ to about 8.0″, from about 3.5″ to about 7.5″, or from about 4.0″ to about 7.0″. In some embodiments, and as shown in FIG. 1, the total width w_{T} is about the same or equal to the third width w₃.

As may be appreciated to those of skill in the art, absorbent article may be of various shapes and sizes to suit the requirements of the user. In at least some embodiments, the first portion 110 is generally elliptical in shape. First portion 110 is typically to be worn to the anterior of the user. In some embodiments according to this disclosure, the first portion 110 provides functionality in providing protection as needed in the event of urinary incontinence.

Absorbent article 100 includes a second portion 130 extending from the connecting portion 120 and having a third width w₃, wherein the third width w₃ is greater than the second width w₂. Second portion 130 is typically to be worn to the posterior of the user. In some embodiments according to this disclosure, the second portion 130 provides functionality in providing protection as needed in the event of anal incontinence. In some embodiments according to this disclosure, the absorbent article 100 may include wherein the second portion 130 is circular and includes a diameter, the diameter ranging from about 3.5″ to about 7.5″. Alternatively, second portion is of an irregular shape symmetrical about the A axis. Second portion 130 includes a width w₃, the width w₃ ranging from about 3″ to about 8″. The width w₃ may be about 3.0″, about 3.5″, about 4.0″, about 4.5″, about 5.0″, about 5.5″, about 6.0″, about 6.5″, about 7.0″, about 7.5″, about 8.0″, in some embodiments, the width w₃ is from about 3.0″ to about 8.0″, from about 3.5″ to about 7.5″, or from about 4.0″ to about 7.0″.

Absorbent article 100 further includes a fastening system 160 arranged on an outer surface 140 of the absorbent article 100 for fixing the absorbent article 100 to an inner surface (not shown) of an article of clothing. The second portion 130 includes a middle section 180 including the longitudinal axis A and having an edge 185 perpendicular to the longitudinal axis A. The second portion 130 further includes that middle section 180 is disposed between first and second wing sections, 190 and 195, the first and second wing sections, 190 and 195, being symmetrical about the longitudinal axis A. In some embodiments according to the present disclosure, the wing sections, 190 and 195, are tapered at the sides as shown in FIG. 1. In some embodiments according to the present disclosure, the edge 185 is flat. In other embodiments according to the present disclosure, the edge 185 is curved. Advantageously, the wing sections 190, 195 having tapered or curved sides extending to the first portion 110 of the absorbent article, which may be circular, keeps the article 100 secured to an undergarment or brief so that the article does not move or shift during wear. As shown in FIG. 2, the wing sections, 190 and 195, include at least a portion of the fastening system 160 connected to the outer surface 140 of absorbent article 100. The fastening system 160 may include adhesives or button or other means positioned on the wing sections 190, 195. The fastening system 160 may further include adhesives or other means positioned on the connecting portion 120 and/or the first portion 110 for further security. Advantageously, the article shape is designed for ease of use, comfort to the wearer, and to function in conjunction with any type of undergarment or brief. In some embodiments, the absorbent article may be wingless.

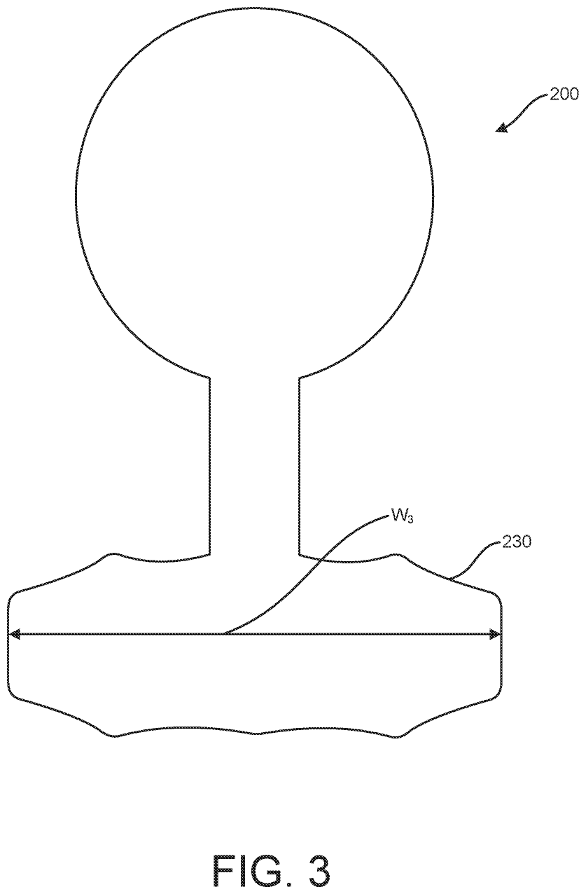

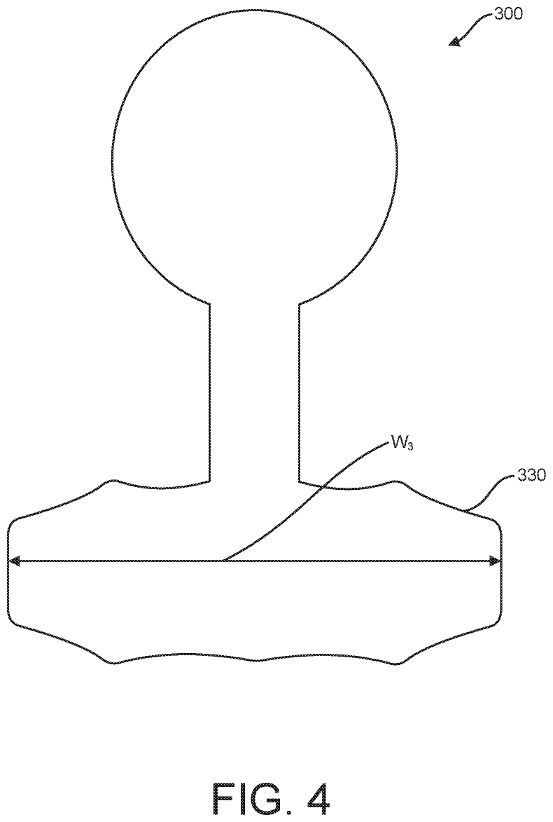

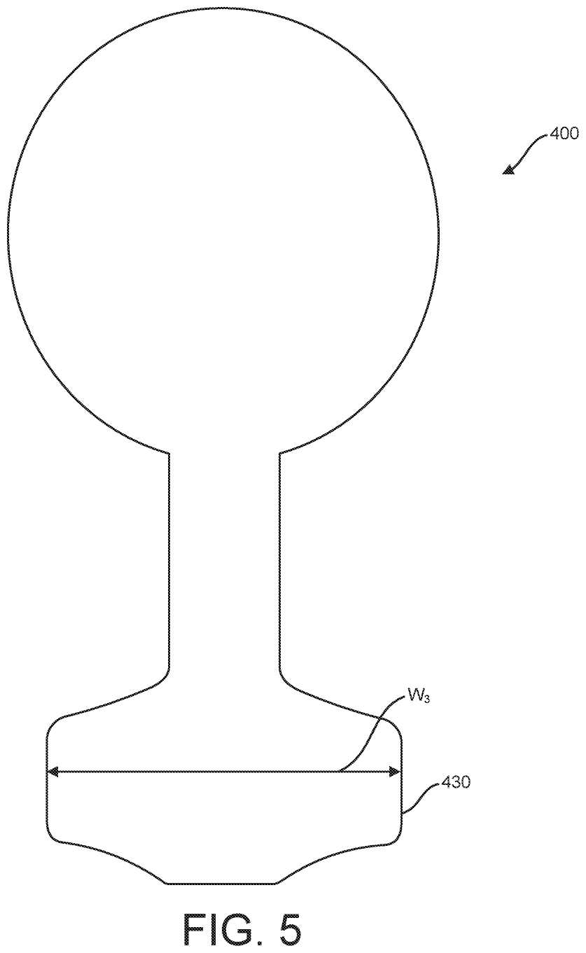

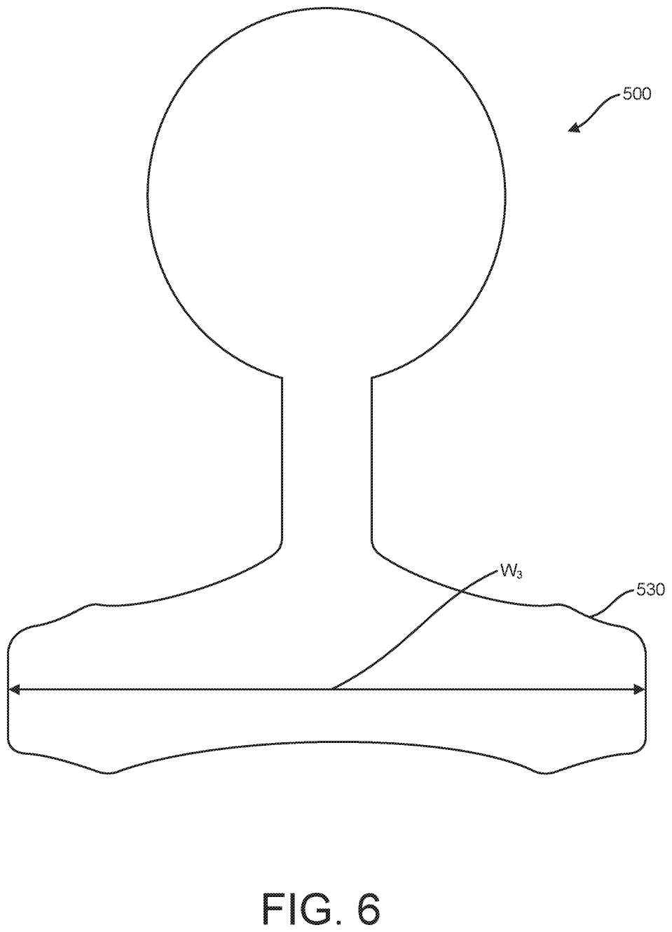

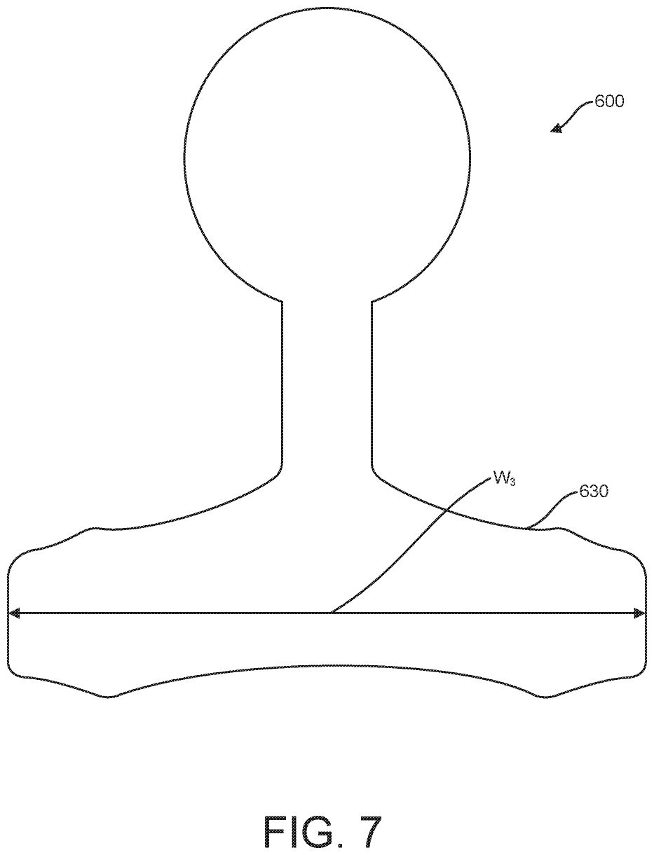

As shown in FIGS. 3 through 7, alternative embodiments of absorbent articles (200, 300, 400, 500, and 600) according to the present disclosure include second portions (230, 330, 430, 530, and 630) of various shapes. Notably, width w₃ of second portions (230, 330, 430, 530, and 630) varies. In some embodiments according to the present disclosure, the second portion includes a modified classic brief shape. In some embodiments according to the present disclosure, the second portion includes a modified high-cut brief shape. In some embodiments according to the present disclosure, the second portion includes a modified hipster brief shape. In some embodiments according to the present disclosure, the second portion includes a modified bikini brief shape. In some embodiments according to the present disclosure, the second portion includes a modified boyshorts brief shape. In some embodiments according to the present disclosure, the second portion includes a modified tanga brief shape.

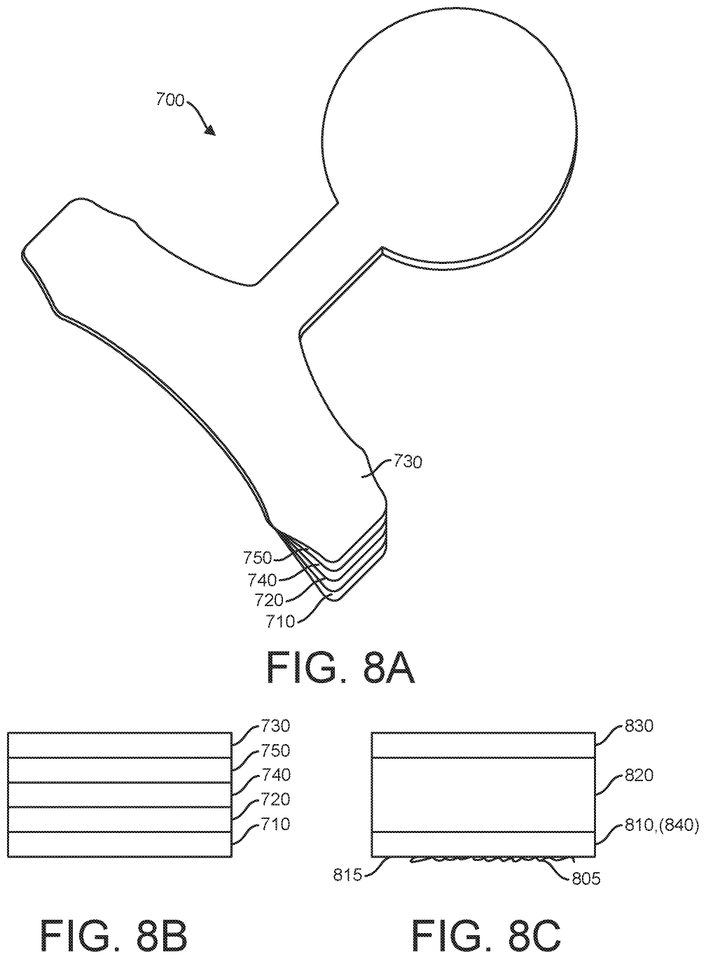

The absorbent articles (200, 300, 400, 500, and 600) according to the present disclosure include at least one material layer. In some embodiments, the absorbent articles (200, 300, 400, 500, and 600) include multiple material layers. A perspective view of an absorbent article 700 according to the present disclosure having multiple material layers is shown in FIG. 8A and a cross-sectional view is illustrated as in FIG. 8B. Absorbent article 700 includes an outer layer 710, which is disposed furthest from the skin of the wearer of the absorbent article. Outer layer 710 is made from a natural breathable fabric chosen from cotton, flannel woven cotton, bamboo, or combinations thereof. Optionally one or more intermediate layers 720 of cotton may be added for comfort and disposed adjacent to the outer layer 710 and/or as an inner layer 730 in contact with the wearer's skin. Inner layer 730 may be made of flannel or cotton. Inner layer 730 may also include a fastening system (not shown) similarly as for fastening system 160 described above. The fastening system for absorbent article 700 may include buttons, snaps, Velcro®, or the like. The buttons or snaps may be of plastic or metal. Absorbent article 700 further includes a wetness protection layer 740, which may be disposed adjacent to the outer layer 710 or to an optional cotton layer 720. Wetness protection layer 740 is made from an impermeable material, which may be a film, chosen from a polyester, a polyethylene terephthalate (PET), a biaxially-oriented polyethylene terephthalate, or combinations thereof. An example of a wetness protection layer 740 is made from Mylar®, a biaxially-oriented polyethylene terephthalate, which is a polyester film made from stretched polyethylene terephthalate (PET) known for its high tensile strength, chemical and dimensional stability, as well as an impermeable moisture barrier. The wetness protection layer 740 has a further advantage of adding rigidity to the absorbent article 700 to prevent the article from sagging or drooping and keeps the product in position. In some embodiments, the wetness protection layer 740 is further layered with an optional odor protection layer 750 adhered thereto. Odor protection layer 750 may be an adhesive layer, such as a disposable, self-adhering charcoal filter, an example of which is Coloplast® Filtrodor. Alternatively, odor protection layer 750 may made a carbon layer, or alternatively made of a material such as cotton impregnated with a carbon media. The at least two layers chosen from outer layer 710, intermediate layer(s) 720, inner layer 730, and combinations thereof are sewn together. Wetness protection layer 740 and optional odor protection layer 750, which are bulkier textiles, are positioned within the device and within the stitching connecting the at least two layers chosen from outer layer 710, one or more intermediate layers 720, inner layer 730, and combinations thereof. The odor protection layer 750 adheres to the wetness protection layer 740 and may be cut to smaller dimensions than the overall device, and therefore need not be sewn. The outer layer 710 is at least sewn to the inner layer 730 with the wetness protection layer 740 and optional odor protection layer 750 disposed there within.

Absorbent article 700 is washable and renewable for reuse. In some embodiments, absorbent article 700 is washed and reused for a time period of about 90 days. In some embodiments, absorbent article 700 is washed and reused for a time period of about 90 days and from about 90 uses to about 180 uses. The life of the article is limited due to possible degradation from repeated usages and washes of the wetness protection layer 740 and/or the optional odor protection layer 750.

Wetness protection layer 740 of absorbent article 700 has a thickness. In some embodiments, the wetness protection layer thickness may be about 0.26 mm, about 0.35 mm, about 0.45 mm, about 0.55 mm, about 0.64 mm, about 0.74 mm, about 0.83 mm, about 0.93 mm, or about 1.02 mm, in some embodiments, the thickness is from about 0.26 mm to about 1.02 mm. Wetness protection layer 740 has a diameter. In some embodiments, the wetness protection layer diameter may be about 2.0″, about 2.5″, about 3.0″, about 3.5″m, or about 4.0″, in some embodiments, the diameter is from about 2.0″ to about 4.0″.

Optional odor protection layer 750 of absorbent article 700 has a thickness. In some embodiments, the odor protection layer thickness may be about 0.10 mm, about 0.25 mm, about 0.50 mm, about 0.75 mm, about 1.00 mm, about 1.25 mm, about 1.50 mm, or about 1.54 mm, in some embodiments, the thickness is from about 0.10 mm to about 1.54 mm. Odor protection layer 750 has a diameter. In some embodiments, the odor protection layer diameter may be about 1.0″, about 1.5″, about 2.0″, about 2.5″, about 3.0″, about 3.5″m, or about 4.0″, in some embodiments, the diameter is from about 1.0″ to about 4.0″.

In other embodiments, absorbent articles (200, 300, 400, 500, and 600) according to the present disclosure are disposable. Absorbent articles 800 is disposable and includes multiple material layers as illustrated in a cross-sectional view shown in FIG. 8C. In some embodiments, the absorbent article 800 may be wingless (not shown). Absorbent article 800 includes an outer layer 810, which is disposed furthest from the skin of the wearer of the absorbent article. Outer layer 810 optionally includes an adhesive 805 on the outer surface 815 so that the disposable absorbent article 800 may be directly positioned and adhered to an inner surface of article of clothing (not shown). Adhesive 805 is chosen from polymer or synthetic resins suitable for adhering to fabric. Outer layer 810 is made from a thin plastic chosen from a polypropylene, a polyethylene, a polyester, a viscose, or combinations thereof. Optionally one or more intermediate layers 820 of wood fiber or polyester fiber may be added for acquiring moisture and pulling the moisture away from the wearer's skin for comfort; layer(s) 820 are disposed adjacent to the outer layer 810. Absorbent article 800 further includes an absorbent core layer 830 chosen from paper pulp, cotton, or combinations thereof. Optionally, absorbent article 800 includes an odor protection layer 840 made of carbon cloth that may be in addition to or replace outer layer 810. The materials selection for disposable absorbent articles 800 may further include one or more layers chosen from outer layer 810, intermediate layer(s) 820, absorbent core layer 830, odor protection layer 840, and combinations thereof including OdorLock Technology™ to incorporate perfume, scents, or the like to mask odors for the disposable absorbent articles 800. The absorbent core layer 830 may further include an ABSORB-LOC® core center to prevent leaks.

The absorbency levels of any of the absorbent articles (200, 300, 400, 500, 600, 700, and 800) may be tailored for very light, light, moderate, and heavy protection levels by adjusting the number of layers and the thickness of the layers accordingly. The thicknesses of the layers levels of any of the absorbent articles (200, 300, 400, 500, 600, 700, and 800) may be tailored to provide the desired absorbency level.

In other embodiments, absorbent articles (200, 300, 400, 500, 600, 700, and 800) according to the present disclosure are made using organic materials wherein the outer layer (e.g., outer layer 710 of absorbent article 700 and/or outer layer 810 of absorbent article 800) is made from cotton fiber. The foregoing discussion has been presented for purposes of illustration and description. The foregoing is not intended to limit the disclosure to the form or forms disclosed herein. In the foregoing Summary for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An absorbent article for use as an undergarment hygiene product, the absorbent article comprising:
a first portion having an elliptical shape and a first width;
a connecting portion extending from the first portion and having a second width;
a second portion extending from the connecting portion and having a third width, the third width greater than the second width; and,
a fastening system arranged on an outer surface of the absorbent article for fixing the absorbent article to an inner surface of an article of clothing.

2. The absorbent article of claim 1, wherein the fastening system includes adhesives.

3. The absorbent article of claim 1, wherein the fastening system includes buttons.

4. The absorbent article of claim 2, wherein the absorbent article is disposable.

5. The absorbent article of claim 3, wherein the absorbent article is renewable.

6. The absorbent article of claim 5, wherein the absorbent article includes at least one material layer, wherein the material layer is chosen from cotton, flannel woven cotton, bamboo, or combinations thereof.

7. The absorbent article of claim 2, wherein the absorbent article includes at least one material layer, wherein the material layer is chosen from a polypropylene, a polyethylene, a polyester, a viscose, or combinations thereof.

8. The absorbent article of claim 1, wherein the connecting portion includes a first length l₁, the first length ranging from 1.5″ to 2.5″.

9. The absorbent article of claim 1, wherein the absorbent article includes a second length l_{T}, the second length ranging from 6″ to 8.5″.

10. The absorbent article of claim 1, wherein the absorbent article includes a width w_{T}, the width ranging from 3″ to 8″.

11. The absorbent article of claim 1, wherein the first portion is circular and includes a diameter, the diameter ranging from 3.5″ to 5.5″.

12. The absorbent article of claim 1, wherein the article of clothing is underpants.

13. An absorbent article for use as an undergarment hygiene product, the absorbent article comprising:
a longitudinal axis,
a first portion having an elliptical shape and a first width symmetrical about the longitudinal axis;
a connecting portion extending from the first portion and having a second width;
a second portion extending from the connecting portion and having a third width, the third width greater than the second width;
a fastening system arranged on an outer surface of the absorbent article for fixing the absorbent article to an inner surface of an article of clothing: and,
wherein the second portion includes a middle section including the longitudinal axis and having an edge perpendicular to the longitudinal axis, the middle section between first and second wing sections, the first and second wing sections symmetrical about the longitudinal axis.

14. The absorbent article of claim 13, wherein the edge is flat or curved.

15. The absorbent article of claim 13, wherein the wing sections include the fastening system.

16. The absorbent article of claim 13, wherein the wing sections are tapered.

17. The absorbent article of claim 13, wherein the second portion includes a modified classic brief shape.

18. The absorbent article of claim 13, wherein the second portion includes a modified high-cut brief shape.

19. The absorbent article of claim 13, wherein the second portion includes a modified hipster brief shape.

20. The absorbent article of claim 13, wherein the second portion includes a modified bikini brief shape.

21. The absorbent article of claim 13, wherein the second portion includes a modified boyshorts brief shape.

22. The absorbent article of claim 13, wherein the second portion includes a modified tanga brief shape.
